SEQUENCE LISTING (5 sequences)

SEQ ID NO: 1 - DNA, 79 bp, Artificial Sequence
  Synthetic oligonucleotide
gggaattcggtaccggatccggtggaggtggaggtggaggtggagactacaaggatgacgatgacaagtaatctagagc

SEQ ID NO: 2 - DNA, 79 bp, Artificial Sequence
  Synthetic oligonucleotide
gctctagattacttgtcatcgtcatccttgtagtctccacctccacctccacctccaccggatccggtaccgaattccc

SEQ ID NO: 3 - DNA, 19 bp, Artificial Sequence
  Synthetic oligonucleotide
gcagattgtactgagagtg

SEQ ID NO: 4 - DNA, 16 bp, Artificial Sequence
  Synthetic olgonucleotide
atggcgggacacctgg

SEQ ID NO: 5 - DNA, 28 bp, Artificial Sequence
  Synthetic oligonucleotide
gggaattcgtttgaatgcatgggagagc

(19) United States
(12) Patent Application Publication
(10) Pub. No.: US 2013/0224833 A1
(43) Pub. Date: Aug. 29, 2013

(54) Method for Preparing and Using Cell Extract Solution for Tubulin-free Cell-free Protein Synthesis and Reagent Kit for Cell-free Protein Synthesis

(71) Applicant: SHIMADZU CORPORATION, (US)

(72) Inventors: Toru EZURE, Osaka (JP); Takashi SUZUKI, Osaka (JP); Rintaro YAMAMOTO, Kyoto (JP); Eiji ANDO, Kyoto (JP)

(73) Assignee: SHIMADZU CORPORATION, Kyoto-shi (JP)

(21) Appl. No.: 13/736,701

(22) Filed: Jan. 8, 2013

(30) Foreign Application Priority Data
Jul. 22, 2010 (JP) 2010-164541

(51) Int. Cl. C12N 9/00

(52) U.S. Cl. CPC C12N 9/00
USPC 435/207; 435/183

(57) ABSTRACT

A method is provided for preparing cell extract solution for cell-free protein synthesis that can effectively remove tubulin protein, a reagent kit for cell-free protein synthesis including the extract solution, and a method for synthesizing cell-free protein using the extract solution. The method for preparing cell extract solution for cell-free protein synthesis includes the steps of: obtaining an extraction treated material of cultured cells by subjecting the cultured cells to an extraction treatment using an extraction buffer; obtaining a reaction mixture by subjecting the extraction treated material to tubulin polymerization reaction and polymerizing the tubulin derived from the culture cells included in the extraction treated material; and preparing the cell extract solution for cell-free protein synthesis by subjecting the reaction mixture to removal of the polymerized tubulin and a buffer exchange.

### TECHNICAL FIELD

The present invention relates to cell-free protein synthesis reaction and, more specifically, to a method for preparing cell extract solution used for protein synthesis in a cell-free system.

### BACKGROUND ART

Cell extract solution for cell-free protein synthesis can be prepared by suspending insect cells in an extraction buffer, then rapidly freezing and thawing them on ice to extract the cell contents which are then centrifuged and subjected to buffer exchange in a desalting column (see Patent Literature 1: U.S. Pat. No. 4,324,730). It is known that cell extract solution for cell-free protein synthesis that is obtained by this method contains β-tubulin (see Non-Patent Literature 1: “Cell-Free Protein Synthesis Reagent Kit, Transdirect Insect Cell,” Experimental Protocol, “Affinity Purification of Synthetic Protein,” p. 3, Shimadzu Corporation).

Heterodimers that are formed by the binding of β-tubulin and α-tubulin are the basic structural units of microtubules. Tubulins undergo repeated polymerization and depolymerization inside a cell and contribute to cell division. Patent Literature 2 (Unexamined Patent Application Publication No. 2002-522747) and Patent Literature 3 (Published Japanese Translation of PCT International Publication for Patent Application No. 2008-522624) disclose that microtubules are stabilized by contact with agents such as Paclitaxel and Paclitaxel analogs. Patent Literature 4 (Published Japanese Translation of PCT International Publication for Patent Application No. 2002-541782) discloses that Taxol binds with β-tubulins that are present in microtubules.

Taxol thus has a mechanism of action of suppressing the multiplication of cancer cells by binding with tubulin and promoting polymerization while inhibiting depolymerization and thus stopping cell division.

A method that is known for studying the genetic information of microtubule associated proteins (MAPs) involves purifying the microtubules (the main component of microtubules is β-tubulin) from cultured insect cells (Drosophila melanogaster) and proteins (MAPs) that are bound to microtubules and then separating the MAPs from the microtubules. Various properties of the MAPs are then studied while identifying the genes of MAPs with a molecular weight of 205 kDa. More specifically, the purification of MAPs is performed by steps that include cell recovery, ultrasonic disintegration, homogenization, centrifugation, polymerization reaction that use Taxol (registered trademark), centrifugation and precipitation (including microtubules bound with MAPs) (see Non-Patent Literature 2: The Journal of Cell Biology, 1986, vol. 102, p. 2076-2087).

### PATENT LITERATURE

Patent Literature 1: U.S. Pat. No. 4,324,730
Patent Literature 2: Unexamined Patent Application Publication No. 2002-522747
Patent Literature 3: Published Japanese Translation of PCT International Publication for Patent Application No, 2008-522624
Patent Literature 4: Published Japanese Translation of PCT International Publication for Patent Application No. 2002-541782

### NON-PATENT LITERATURE

Non-Patent Literature 1: “Cell-Free Protein Synthesis Reagent Kit, Transdirect Insect Cell,” Experimental Protocol, “Affinity Purification of Synthetic Protein,” p. 3, Shimadzu Corporation
Non-Patent Literature 2: Lawrence S. B. G., R. A. Laymon, J. R. Mclntoshi, “A Microtubule-Associated Protein in Drosophila Melanogaster: Identification, Characterization, and Isolation of Coding Sequences), “The Journal of Cell Biology,” 1986, vol. 102, p. 2076-2087

### SUMMARY OF THE INVENTION

Cell extract solution for cell-free protein synthesis that is obtained by the method described in Patent Literature 1 (U.S. Pat. No. 4,324,730) contains large quantities of tubulin that are present in the cells used as the derivation source. The proteins that are synthesized by cell-free protein synthesis using the extract solution may be of a type that non-specifically binds to tubulin protein that is included in the extract solution. This means that the presence of tubulin proteins in the extract solution may hinder purification or activity measurement of the proteins that are synthesized.

It is therefore the object of the present invention to provide a method for the preparation of cell extract solution for cell-free protein synthesis, which also effectively removes tubulin proteins. It is a further object of the present invention to provide a reagent kit for cell-free protein synthesis, which also includes cell extract solution for cell-free protein synthesis that is obtained using the afore-described method. Still furthermore, it is the object of the present invention to provide a cell-free protein synthesis method that allows proteins to be obtained with good purification efficiency.

The present inventors discovered that the afore-described objects of the present invention can be achieved by extracting cell contents from cultured insect cells, then performing tubulin polymerization reaction before buffer exchange, and then precipitating the tubulin as polymers. This discovery led to the completion of the present invention.

The present invention includes the following inventions.

(1) A method for the preparation of cell extract solution for cell-free protein synthesis including:

a step for obtaining extraction treated material of cultured cells by subjecting cultured cells to an extraction treatment using an extraction buffer;

a step for obtaining a reaction mixture by polymerizing tubulin derived from the cultured cells included in the extraction treated material by subjecting the extraction treated material to a tubulin polymerization reaction; and

a step for obtaining cell extract solution for cell-free protein synthesis by subjecting the reaction mixture to removal of polymerized tubulin and buffer exchange.

To explain, with the present invention, after the cultured cells are extracted, tubulin polymerization reaction is performed before a buffer exchange. Also, with the present invention, polymerized tubulin (tubulin polymers) is removed, and cell extract solution for cell-free protein synthesis are prepared from the liquid component following the removal of the tubulin polymers.

(2) The method described in (1) wherein, in the afore-described step for obtaining extraction treated material of cultured cells, extraction mixture that is obtained by the extraction treatment is subjected to centrifugation under the conditions of 10,000×g to 50,000×g for 1 to 60 minutes after the extraction treatment, and a supernatant that is obtained by the centrifugation is obtained as the extraction treated material of cultured cells.

In addition to the method described in (2) above, the present invention also includes the following methods.

The method described in (1) wherein, in the step for obtaining the extraction treated material of cultured cells, the extraction mixture that is obtained by the extraction treatment is not separated after the extraction treatment, and the extraction mixture is obtained as the extraction treated material of culture cells.

In the afore-described step for obtaining the extraction treated material of cultured cells, the extraction mixture that is obtained by the extraction treatment is subjected to a separation treatment twice after the extraction treatment and the supernatant that is obtained by the two separation treatments is obtained as the extraction treated material of cultured cells.

(3) The method described in (1) or (2) wherein the extraction is performed by rapidly freezing the cultured cells that are suspended in the extraction buffer and thawing the frozen cultured cells.
(4) The method described in any one of (1) through (3) wherein the tubulin polymerization reaction is performed using taxane compounds and/or epothilone compounds.
(5) The method described in any one of (1) through (4) wherein the cultured cells are cultured insect cells.
(6) The method described in (5) wherein the cultured insect cells are Trichoplusia ni ovum-derived cultured cells and/or Spodoptera frugiperda ovary cell-derived cultured cells.

FIG. 1 shows a simplified flowchart of one mode for performing the preparation method according to the present invention. However, the present invention is not limited to this mode.

(7) A reagent kit for cell-free protein synthesis that includes cell extract solution for cell-free protein synthesis wherein the cell extract solution is prepared using a method described in any one of (1) through (6).
(8) A method for cell-free protein synthesis that uses cell extract solution for cell-free protein synthesis wherein the cell extract solution is prepared using a method described in any one of (1) through (6).

“Extraction” is an operation by which cells are suspended in an “extraction buffer” and are ruptured to expose the cell contents in the extraction buffer. Extracted materials from cultured cells are the exposed cell contents and may also refer in particular to components of cell contents that are used for protein synthesis.

“Extraction treated material” is, at the least, the material that is obtained by “extraction treatment” and is subjected to a “tubulin polymerization reaction.” Examples of “extraction treated material” include “extraction mixture” itself and also “supernatants” that are obtained from an extraction mixture.

As for “extraction mixtures,” extracted materials from cultured cells and other components (which should be ultimately removed such as impurities from cells) are included in the “extraction buffer.”

As for “supernatants,” extracted materials from cultured cells are included in the “extraction buffer.”

A “reaction mixture” is obtained from a “tubulin polymerization reaction.” A “reaction mixture” includes “polymerized tubulin” (sometimes referred to as tubulin polymers), extracted materials from cultured cells and other components that are used for the polymerization.

“Cell extract solution for cell-free protein synthesis” is obtained by removing “polymerized tubulin” from a “reaction mixture” and then performing a buffer exchange.

The present invention provides a method for the preparation of cell extract solution for cell-free protein synthesis that allows tubulin protein to be effectively removed. Furthermore, the present invention provides a reagent kit for cell-free protein synthesis that includes cell extract solution obtained by the aforesaid method for cell-free protein synthesis. Still furthermore, the present invention provides a method for cell-free protein synthesis that allows synthetic protein to be obtained with high purification efficiency.

With the method of preparation of cell extract solution for cell-free protein synthesis according to the present invention, tubulin alone can be specifically removed. Furthermore, the removal of tubulin has almost no effect on the protein synthesis ability of the extract solution.

Because of this, even with proteins whose affinity purification is difficult when the protein is synthesized using extract solution that had been prepared using conventional methods, when the proteins are synthesized using extracted materials that had been prepared using the preparation method of the present invention, the purification efficiency of the same proteins are dramatically increased. The explanation for this is that when extract solution that have been prepared using conventional methods are used, the non-specific binding of tubulin masks the affinity tag of synthetic proteins, worsening the binding efficiency of the synthetic proteins to the affinity support. In contrast to this, when extract solution that have been prepared using the method of the present invention are used, the absence of tubulin results in an effective exposure of the affinity tags of the synthetic proteins, dramatically increasing the binding efficiency to the affinity support.

### BRIEF DESCRIPTION OF THE DRAWINGS

FIG. 1 is a simplified flowchart showing an example of one mode of the preparation method according to the present invention.

FIG. 2 are electrophoresis charts that show the results of the confirmation of tubulin removal in the cell extract solution for cell-free protein synthesis is according to the present invention obtained as Embodiment 1. A comparison is made as Embodiment 2 against cell extract solution for cell-free protein synthesis obtained as Comparison Example 1 (preparation that does not perform the tubulin removal step). The arrow identifies the location of the tubulin band.

FIG. 3 shows the calibration curve prepared in Embodiment 3 for measuring protein synthesis ability.

FIG. 4 shows the results (Embodiment 4) of affinity purification of cell-free protein synthesis using cell extract solution for cell-free protein synthesis according to Embodiment 1 of the present invention and the affinity purification results (Comparison Example 3) of cell-free protein synthesis using conventional cell extract solution for cell-free protein synthesis of Comparison Example 1. The arrow identifies the location of the band for the synthesized and purified target protein (OCT4).

### DETAILED DESCRIPTION OF THE INVENTION

### 1. Cultured Cells

With the present invention, no limitation is imposed on cultured cells that are used as the material for cell extract solution for cell-free protein synthesis so long as they are eukaryotic cells, Cultured cells that have been conventionally used for the preparation of cell extract solution for cell-free protein synthesis can be used. Examples of cultured cells that can be used include insect cells, mammalian animal cells (human derived cells, Chinese hamster ovary (CHO) derived cells, HeLa cells, etc.) and antibody producing hybridomas.

With the present invention, it is preferable for the cultured cells to be cultured insect cells. Further description is provided herein below regarding cultured insect cells, but those skilled in the art can suitably prepare and use other types of cultured cells.

The insect cell to be used in the present invention is not subject to any particular limitation. For example, cells derived from insects of Lepidoptera, Orthoptera, Diptera, Hymenoptera, Coleoptera, Coleoptera, Neuroptera, Hemiptera and the like can be used. Of these, cells derived from insects of Lepidoptera, Hemiptera and the like are preferable, because many culture cell lines thereof have been established. Furthermore, the insect cell in the present invention may be a cell derived from any tissue, and, for example, blood cell, gonad-derived cell, fat body-derived cell, embryo-derived cell, hatch larva-derived cell and the like can be used without any particular limitation. Of these, gonad-derived cell, which is considered to have high protein production capability, is preferably used. Particularly, use of High Five (manufactured by Invitrogen), which is a cell derived from the ovum of Trichoplusia ni or Sf21 (manufactured by Invitrogen), which is a cell derived from Spodoptera frugiperda ovary cell, as an insect cell is preferable, because they have high protein synthesis capability in a cell-system and can be cultured in a serum-free medium. In the present invention, the cell is not limited to an insect cell derived from a single tissue of a single species of insect, and it may be derived from plural kinds of tissues of a single species of insect, or a single kind of tissue of plural species of insects, or derived from plural kinds of tissues of plural species of insects.

With the preparation method of the present invention, if cultured insect cells are used, the cultured cells may be directly subjected to the extraction step described below. However, even though this is not a particular requirement, a washing step may be performed before the extraction step. For example, before the extraction step further described below, the insect cells may be washed with a wash solution having the same composition as the extraction solution further described below except for not containing a protease inhibitor and glycerol. Washing with a wash solution includes addition of the wash solution to an insect cell and centrifugation thereof (e.g., 700×g, 10 minutes, 4° C.). The amount of the wash solution to be used for the washing is preferably 5 mL to 100 mL, more preferably 10 mL to 50 mL, per 1 g (wet weight) of insect cells, for complete removal of the medium. The frequency of washing is preferably 1 to 5 times, more preferably 2 to 4 times.

In addition, while the amount of the cultured insect cells to be subjected to the preparation method of the present invention is not particularly limited, it is preferably 0.1 g to 5 g, more preferably 0.5 g to 2 g, per 1 mL of the extract solution, to maintain optimum extraction efficiency.

### 2. Preparation of Extraction Treated Materials

### 2-1. Extraction

The extraction treatment on the cultured cells is performed by rupturing the cultured cells. No particular limitation is imposed on conventional methods that use an extraction buffer, and those skilled in the art are free to make an appropriate selection. Examples of rupturing methods include suspending cultured cells in an extraction buffer and then freezing and thawing them or mashing them in a mortar with pestle, or suspending cultured cells in an extraction buffer and optionally further freezing them and then rupturing them using a Dounce homogenizer or glass beads.

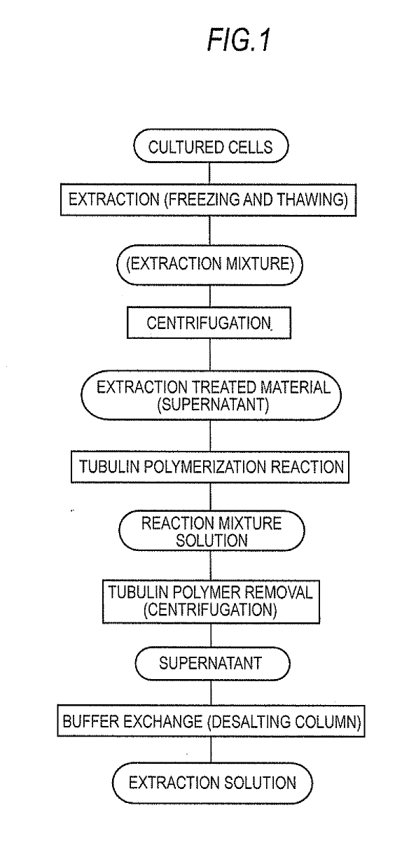

With the preparation method of the present invention, it is preferable to perform the extraction using a method wherein cultured cells suspended in an extraction buffer are rapidly frozen (e.g., in the manner shown in FIG. 1).

With this method, the phrase “rapidly frozen” means that cultured cells are frozen in no longer than 10 seconds, preferably no longer than 2 seconds, after subjecting the cells to a freezing treatment. When the cultured cells are not rapidly frozen in the present invention, the components essential for protein synthesis may be inactivated, and the extraction efficiency from the cells may decrease. The temperature to be used for rapidly freezing is generally not higher than −80° C., preferably not higher than −150° C. This is because the components essential for protein synthesis tend to become inactivated, and protein synthesis ability tends to be degraded when the cells are rapidly frozen at a temperature exceeding −80° C.

The above-mentioned rapid freezing of the cultured cells can be achieved by, for example, using an inert gas such as liquid nitrogen, liquid helium and the like. It is preferable to use liquid nitrogen because it is easily available and economical.

The extraction from the afore-described rapidly frozen cultured cells is completed by thawing (e.g., in the manner shown in FIG. 1). Particularly, in the case of animal cell-derived cultured cells (e.g., cultured insect cells), the use of this method is preferable since cells are easily ruptured. Furthermore, the extract solution that is obtained by extraction based on freezing and thawing contains components essential for protein synthesis in their active state and is also preferable for providing an extract solution with a high protein synthesis ability.

The thawing of the afore-described rapidly frozen cultured cells can be realized by thawing in water bath or ice-water bath at −10° C. to 20° C. by leaving the cells to stand at room temperature (25° C.) and the like. To prevent inactivation of the components essential for protein synthesis and to surely prevent degradation of protein synthesis ability, the cells are preferably thawed in water bath or ice-water bath at 0° C. to 20° C. (particularly 4° C. to 10° C.).

With the present invention, the extraction mixture itself that is obtained from the afore-described extraction or the supernatant that is obtained as described before from the extraction mixture can become the extraction treated material that is subjected to the tubulin polymerization reaction further described below.

### 2-2. Obtaining the Supernatant

The extraction mixture that is obtained from the afore-described extraction may be subjected to centrifugation prior to tubulin polymerization reaction further described below to remove cellular residues including nuclei and membrane components. Centrifugation allows supernatants to be obtained from the extraction mixture, and the supernatant that is collected can be used as the extraction treated material that is subjected to the tubulin polymerization reaction.

No particular limitation is imposed on the specific conditions of the centrifugation as long as cellular residues including the nuclei and membrane components are separated and the protein synthesis ability of the supernatant is appropriately preserved. Specifically, conditions such as 10,000×g to 50,000×g and 1 to 60 minutes can be used. If the condition falls short of the above range, there is a tendency for cellular residues including the nuclei and membrane components to remain in the supernatant, and if the above conditions are exceeded, there is a tendency for the protein synthesis ability of the supernatant to decrease. An example of a temperature condition that can be used for the centrifugation is 0 to 10° C.

If centrifugation is performed under the above conditions, the centrifugation may be performed once or twice. However, from the perspective of protein synthesis activity, there are times when the preference is to perform centrifugation once.

### 2-3. Extraction Buffer

The extraction buffer to be used for the afore-described extraction is not particularly limited but preferably contains at least a protease inhibitor. When an extraction buffer containing a protease inhibitor is used, the activity of the protease contained in extracted materials derived from cultured cells is inhibited, thereby preventing undesired decomposition of the active proteins in the extraction mixture caused by the protease. The result is that the protein synthesis ability of the cell extract solution for cell-free protein synthesis is effectively and advantageously exhibited.

The above-mentioned protease inhibitor is not particularly limited as long as it can inhibit the activity of protease, and, for example, phenylmethanesulfonyl fluoride (hereinafter sometimes referred to as “PMSF”), aprotinin, bestatin, leupeptin, pepstatin A, E-64. (L-trans-epoxysuccinyl-leucylamido-4-guanidinobutane), ethylenediaminetetraacetic acid, phosphoramidon and the like can be used. Since extracted materials derived from cultured cells may contain serine protease, the use of PMSF, which works as an inhibitor having high specificity to serine protease, is preferable among those mentioned above. It is possible to use not only one kind of protease inhibitor but also a mixture (protease inhibitor cocktail) of several kinds of protease inhibitors.

The protease inhibitor content in the extraction buffer is free of any particular limitation but is preferably 1 μM to 50 mM, more preferably 0.01 mM to 5 mM, because decomposition of the enzymes necessary for the action of the present invention can be preferably inhibited. This is because, when the protease inhibitor content is less than 1 μM, the decomposition activity of protease often cannot be suppressed sufficiently, and when the protease inhibitor content exceeds 50 mM, the protein synthesis reaction tends to be inhibited.

The extraction buffer to be used for the present invention preferably contains, in addition to the above-mentioned protease inhibitor, at least a potassium salt, a magnesium salt, dithiothreitol, chelating agent and a buffer.

The above-mentioned potassium salt is free of any particular limitation as long as it does not inhibit the action of the present invention, and can be used in a general form, such as potassium acetate, potassium carbonate, potassium hydrogen carbonate, potassium chloride, dipotassium hydrogen phosphate, dipotassium hydrogen citrate, potassium sulfate, potassium dihydrogen phosphate, potassium iodide, potassium phthalate and the like, with preference given to potassium acetate. Potassium salt acts as a cofactor in the protein synthesis reaction.

The content of the potassium salt in the extraction buffer is free of any particular limitation, but from the aspect of storage stability, it is preferably 10 mM to 500 mM, more preferably 50 mM to 300 mM, in the case of a monovalent potassium salt, such as potassium acetate and the like. This is because when the content of the potassium salt is less than 10 mM or more than 500 mM, the components essential for protein synthesis tend to become unstable.

The above-mentioned magnesium salt is free of any particular limitation as long as it does not inhibit the action of the present invention, and can be used in a general form such as magnesium acetate, magnesium sulfate, magnesium chloride, magnesium citrate, magnesium hydrogen phosphate, magnesium iodide, magnesium lactate, magnesium nitrate, magnesium oxalate and the like, with preference given to magnesium acetate. Magnesium salt also acts as a cofactor in the protein synthesis reaction.

The content of the magnesium salt in the extraction buffer is free of any particular limitation, but from the aspect of storage stability, it is preferably 0.1 mM to 10 mM, more preferably 0.5 mM to 5 mM, in the case of a divalent salt, such as magnesium acetate and the like. This is because when the content of the magnesium salt is less than 0.1 mM or more than 10 mM, the components essential for protein synthesis tend to become unstable.

The above-mentioned dithiothreitol (“DTT”) is added for prevention of oxidization and is preferably contained in an amount of 0.1 mM to 10 mM, more preferably 0.5 mM to 5 mM, in the extraction buffer. This is because when the content of DTT is less than 0.1 mM or more than 10 mM, the components essential for protein synthesis tend to become unstable.

The above-mentioned chelating agent is free of any particular limitation as long as it does not inhibit the action of the present invention and can be used in a general form such as ethylene glycol tetraacetic acid (EGTA), ethylenediaminetetraacetic acid (EDTA) and the like, with preference given to EGTA. A chelating agent inhibits the depolymerization of tubulin by chelating calcium, which promotes the depolymerization of tubulin.

The content of the chelating agent in the extraction buffer is free of any particular limitation, but from the aspect of storage stability, it is preferably 0.2 mM to 20 mM, more preferably 1 mM to 10 mM, in the case of, for example, EGTA. This is because when the content of the chelating agent is less than 0.2 mM or more than 20 mM, tubulin tends to be depolymerized, and the components essential for protein synthesis tend to become unstable.

In an extraction treated material obtained by extraction using an extraction buffer, the above-mentioned buffer is added for prevention of denaturation of extracted materials caused by a rapid change in pH caused by, for example, addition of an acidic or basic substance and the like. Such buffer is free of any particular limitation, and, for example, HEPES-KOH, Tris-HCl, acetic acid-sodium acetate, citric acid-sodium citrate, phosphoric acid, boric acid, MES, PIPES and the like can be used.

The buffer is preferably one that maintains the pH of the extraction treated material at 4 to 10, more preferably pH of 6.5 to 8.5. When the pH of the extract solution is less than 4 or more than 10, the components essential for the reaction of the present invention may be denatured. From this aspect, the use of HEPES-KOH (pH 6.5 to 8.5) is particularly preferable among the above-mentioned buffers.

While the content of the buffer in the extraction buffer is free of any particular limitation, it is preferably 5 mM to 200 mM, more preferably 10 mM to 100 mM, to maintain preferable buffer capacity. When the content of the buffer is less than 5 mM, pH tends to change radically when an acidic or basic substance is added, which in turn may cause denaturation of the extracted material. When the content of the buffer exceeds 200 mM, the salt concentration becomes too high, and the components essential for protein synthesis tend to become unstable.

In addition to the afore-described composition, the extraction buffer may also contain glycerol. The use of such extraction buffer is preferable since it provides a cell extract solution for cell-free protein synthesis having improved protein synthesis ability.

While the amount of glycerol that is added is free of any particular limitation, for effective manifestation of the effect of the afore-described improved protein synthesis ability, it is preferably added in a proportion of 5 (v/v) % to 80 (v/v) %, more preferably 10 (v/v) % to 50 (v/v) %.

### 2-4. Extraction Treated Material of Cultured Cells

By using the afore-described extraction buffer, the extraction treated material that is obtained by the extraction treatment can be prepared to have, at the least, the following composition. To explain, the extracted material derived from the cultured cells will contain: protein concentration of 1 mg/mL to 200 mg/mL, preferably 10 mg/mL to 100 mg/mL; potassium salt (e.g., potassium acetate) of 10 mM to 500 mM, preferably 50 mM to 300 mM; magnesium salt (e.g., magnesium acetate salt) of 0.1 mM to 10 mM, preferably 0.5 mM to 5 mM; DTT of 0.1 mM to 10 mM, preferably 0.5 mM to 5 mM; chelating agent (e.g., EGTA) of 0.2 mM to 20 mM, preferably 1 mM to 10 mM; protease inhibitor (e.g., PMSF) of 1 μM to 50 mM, preferably 0.01 mM to 5 mM; buffer (e.g., HEPES-KOH (pH 6.5 to 8.5)) of 5 mM to 200 mM, preferably 10 mM to 100 mM; and glycerol of 5 (v/v) % to 80 (v/v) %, preferably 10 (v/v) % to 50 (v/v) %.

### 3. Tubulin Polymerization

The extraction treated material of cultured cells is subjected to tubulin polymerization reaction, and provides a reaction mixture that includes polymerized tubulin.

### 3-1. Polymerization Reagent

No particular limitations are imposed on the tubulin polymerization reagent so long as the substance (antimitotic agent) has the action of binding to tubulin, promoting tubulin polymerization (depolymerization inhibition) and stabilizing the microtubules. Examples of such substances include taxane compounds and epothilone compounds.

Taxane compounds are compounds whose basic skeleton is a taxane ring (tricyclo [9.3.1.0^{3,8}] pentadecane). Examples include taxane-based anti-cancer drugs like paclitaxels (e.g., Taxol (registered trademark)) and docetaxels (e.g., Taxotere (registered trademark)).

Epothilone compounds are compounds that have a 16-membered ring macrolide structure and a thiazole ring side chain. Examples include epothilone based anti-cancer drugs such as epothilone A, its analogs, epothilone B and its derivatives (e.g., ixabepilone).

The afore-described polymerization reagent may be present in a polymerization reaction system in amounts such as 0.1 μM to 500 μM, preferably 5 μM to 100 μM, for example, 20 μM. When the amount is less than the above range, there is a tendency for tubulin to not polymerize, and when the above range is exceeded, there is a tendency for the ingredients essential for protein synthesis to become unstable.

Furthermore, in addition to the afore-mentioned reagent, guanosine triphosphate (GTP) is used. In the tubulin polymerization step, tubulin dimers with bound GTP are polymerized. The concentration of GTP in the polymerization reaction system may be 0.01 mM to 50 mM, preferably 0.5 mM to 10 mM, for example, 2 mM. When the concentration is less than the above range, there is a tendency for tubulin to not polymerize, and when the above range is exceeded, there is a tendency for the ingredients essential for protein synthesis to become unstable.

The extraction buffer that is used in the extraction treatment contains in advance components that are involved in tubulin polymerization. When the tubulin polymerization reaction system is created, it is possible for the reaction system to contain such components in amounts sufficient for the polymerization reaction (e.g., in the case of GTP or Taxol, in amounts necessary to satisfy the afore-described concentrations). If that is the case, there is no need during the tubulin polymerization step to again add such components that are involved in the tubulin polymerization reaction.

However, even if the extraction buffer that is used in the extraction treatment may contain in advance components that are involved in tubulin polymerization, the components may not be present in sufficient amounts when the tubulin polymerization reaction system is created. If that is the case, additional components to make up for the shortage may be added during the tubulin polymerization step so that sufficient amounts of the components are present in the reaction system (e.g., in the case of GTP or Taxol, in amounts necessary to satisfy the afore-described concentrations),

In the polymerization reaction system, the content of the afore-described extraction treated material of cultured cells may be adjusted so that it is 50 (v/v) % to 99 (v/v) %, preferably 80 (v/v) % to 99 (v/v) %. There is a need to hold down the dilution as much as possible so that the protein synthesis ability is not decreased. If the content is less than the above range, there is a tendency for the protein synthesis ability to decrease.

The polymerization reaction temperature can be room temperature. More specifically, the polymerization reaction can be carried out at 10 to 40° C., preferably 15 to 35° C. When the temperature exceeds the above range, there is a tendency for the protein synthesis ability to decrease, and when the temperature is below the above range, there is a tendency for tubulin polymerization reaction to not proceed fully.

Also, the polymerization reaction time can be set to be 5 to 120 minutes, preferably 20 to 60 minutes. When the above range is exceeded, there is a tendency for the protein synthesis ability to decrease, and when the above range is not met, there is a tendency for the tubulin polymerization reaction to not proceed fully.

### 4. Tubulin Polymer Removal and Buffer Exchange

The reaction mixture that is obtained by the tubulin polymerization reaction is subjected to tubulin polymer removal and buffer exchange. What results is a cell extract solution for cell-free protein synthesis.

### 4-1. Tubulin Polymer Removal

Tubulin polymers can be removed by performing a separation and then collecting the supernatant from the reaction mixture. No particular limitation is imposed on the specific method of separation, and those skilled in the art can select the separation method that is appropriate in the field of preparation of cell extract solution for cell-free protein synthesis. However, a preferable method is centrifugation (e.g., following the mode shown as an example in FIG. 1). More specifically, conditions that are generally used in this field can be used (e.g., 10,000×g to 50,000×g, 0° C. to 30° C., 10 minutes to 60 minutes).

No particular limitation is imposed on the number of times that the separation is performed, and it can be once or twice. However, from the perspective of the protein synthesis ability of the cell extract solution for cell-free protein synthesis, there are times when one separation is preferable.

### 4-2. Buffer Exchange

The supernatant that is obtained by the afore-described removal is subjected to buffer exchange. The buffer exchange removes low molecular weight impurities and provides a cell extract solution for cell-free protein synthesis.

No particular limitation is imposed on the buffer that is used for the buffer exchange, and those skilled in the art may make a suitable selection. Examples of buffers that may be used include those that contain 10 mM to 100 mM of buffer (e.g., HEPES-KOH) (pH 6.5 to 8.5), 50 mM to 300 mM of potassium salt (e.g., potassium acetate), 0.5 mM to 5 mM of magnesium salt (magnesium acetate), 0.5 mM to 5 mM of DTT, 1 (v/v) % to 20 (v/v) % of glycerol and 0.01 mM to 5 mM of protease inhibitor (e.g., PMSF).

No particular limitation is imposed on the specific method of buffer exchange, and an appropriate method can be selected by those skilled in the art.

With the present invention, gel filtration is preferable. A desalting column can be used for the gel filtration (in the mode shown in FIG. 1). An example of one that can be preferably used is PD-10 (manufactured by GE Healthcare Biosciences). According to conventional methods, the column is equilibrated with a buffer solution for gel filtration, and a specimen is fed and eluted using the above-mentioned buffer solution for gel filtration. As the above-mentioned buffer solution for gel filtration, conventionally known buffer solutions having appropriate compositions can be used without any particular limitation. As an example, an exchange buffer having the afore-described composition can be used as the gel filtration buffer solution.

Furthermore, with the present invention, extract solution can be concentrated after the buffer exchange. For example, fractions having a high absorbance can be collected from the filtrate after the afore-described gel filtration to obtain extract solution having a high concentration of extracted materials from cultured cells, which is preferable from the perspective of protein synthesis ability. The filtrate obtained by gel filtration may be fractionated into 0.1 mL to 1 mL fractions as one unit of fraction as in general gel filtration, but 0.4 mL to 0.6 mL is preferably used as one unit of fraction from the perspective of efficiently obtaining a fraction having high protein synthesis ability.

It is preferable to separate a fraction (with high absorbance) having an absorbance at 280 nm of not less than 10, preferably not less than 30, from the fractions. A fraction that is obtained in this way can be used as the cell extract solution for cell-free protein synthesis.

### 5. Cell Extract Solution for Cell-Free Protein Synthesis

The cell extract solution from cultured cells for cell-free protein synthesis, which is prepared according to the method of the present invention, preferably contains extracted materials derived from cultured cells in a protein concentration of 1 mg/mL to 200 mg/mL, more preferably 10 mg/mL to 100 mg/mL. When the extracted material content measured in protein concentration is less than 1 mg/mL, the concentration of the components essential for achieving the effects of the present invention becomes low, and this raises the risk of the synthesis reaction not being performed sufficiently. When the extracted material content measured in protein concentration exceeds 200 mg/mL, the extract solution itself becomes highly viscous, making operations difficult.

The content of extracted materials derived from cultured cells in the extract solution can be determined by measuring the protein concentration, for example, by using a BCA protein assay kit (manufactured by Pierce). For example, a sample (0.1 mL) was added to a reaction reagent (2 mL) and was reacted at 37° C. for 30 minutes. Absorbance at 562 nm was measured using a spectrophotometer (Biospec-mini manufactured by Shimadzu Corporation). Bovine serum albumin (BSA) was used as a control and a calibration curve was drawn. Protein concentration can be measured using a method such as this.

The cells from which the extracted materials that are contained in the extract solution are derived can be determined by, for example, base sequence analysis of ribosomal RNA in the extract solution.

The extract solution of the present invention is preferably realized to contain the extracted materials derived from cultured cells in a protein concentration of 10 mg/mL to 100 mg/mL, concurrently with 50 mM to 300 mM of potassium salt (e.g., potassium acetate), 0.5 mM to 5 mM of magnesium salt (e.g., magnesium acetate), 0.5 mM to 5 mM of DTT, 0.01 mM to 5 mM of protease inhibitor (e.g., PMSF) and 10 mM to 100 mM of buffer (e.g., HEPES-KOH (pH 6.5-8.5)). Furthermore, the extract solution preferably contains glycerol in a proportion of 1 (v/v) % to 20 (v/v) %. When the content exceeds the above range, protein synthesis ability tends to decrease, and when the content falls short of the above range, the storage stability of the extracted materials tends to deteriorate. The glycerol concentration in the extract solution can become lower than the glycerol concentration in the extraction treated material.

### 6. Cell-Free Protein Synthesis

The cell extract solution for cell-free protein synthesis according to the present invention can be prepared as a reaction solution for cell-free protein synthesis by adding additives that are involved in cell-free protein synthesis. No particular limitations are imposed on the additives, and additives that are generally and conventionally used in the field of protein synthesis in cell-free systems can be used.

The above-mentioned reaction solution for cell-free protein synthesis is preferably prepared in such a manner that the extract solution of the present invention is contained in a range of 10 (v/v) % to 80 (v/v) %, particularly 30 (v/v) % to 60 (v/v) %.

In other words, it is preferably prepared in such a manner that the content of the extracted materials derived from insect cells in the entire reaction solution is 0.1 mg/mL to 160 mg/mL, more preferably 3 mg/mL to 60 mg/mL. When the extracted material content is less than 0.1 mg/mL or above 160 mg/mL as measured in protein concentration, the rate of synthesis of the object protein may become lower.

Generally, the above-mentioned reaction solution contains, as components other than the above-mentioned extract solution, at least potassium salt, magnesium salt, DTT, adenosine triphosphate, guanosine triphosphate, creatine phosphate, creatine kinase, amino acids, RNase inhibitor, tRNA, exogenous mRNA and buffer. This realizes a reaction solution for cell-free protein synthesis with the advantage of being able to synthesize large amounts of protein in a short amount of time.

As the potassium salt in the reaction solution, various potassium salts described above as a component of extract solution, preferably potassium acetate, can be preferable used. The potassium salt is preferably contained in a proportion of 10 mM to 500 mM, more preferably 50 mM 150 mM, from the same perspective as the potassium salt in the aforementioned extract solution.

As the magnesium salt in the reaction solution, various magnesium salts described above as a component of extract solution, preferably magnesium acetate, can be preferably used. The magnesium salt is preferably contained in a proportion of 0.1 mM to 10 mM, more preferably 0.5 mM to 3 mM, from the same perspective as the magnesium salt in the aforementioned extract solution.

DTT is preferably contained in the reaction solution in a proportion of 0.1 mM to 10 mM, more preferably 0.2 mM to 5 mM, from the same perspective as DTT in the aforementioned extract solution.

The adenosine triphosphate (“ATP”) is preferably contained in the reaction solution in a proportion of 0.01 mM to 10 mM, more preferably 0.1 mM to 5 mM, in view of the rate of protein synthesis. When ATP is contained in a proportion of less than 0.01 mM or more than 10 mM, the synthesis rate of the protein tends to become lower.

The guanosine triphosphate (“GTP”) is preferably contained in the reaction solution in a proportion of 0.01 mM to 10 mM, more preferably 0.1 mM to 5 mM, in view of the rate of protein synthesis. When GTP is contained in a proportion of less than 0.01 mM or more than 10 mM, the synthesis rate of the protein tends to become lower.

The creatine phosphate in the reaction solution is a component for continuous synthesis of protein and is added for regeneration of ATP and GTP. The creatine phosphate is preferably contained in the reaction solution in a proportion of 1 mM to 200 mM, more preferably 10 mM to 100 mM, in view of the rate of protein synthesis. When creatine phosphate is less than 1 mM, sufficient amounts of ATP and GTP may not be regenerated easily. As a result, the rate of protein synthesis tends to become lower, and when creatine phosphate exceeds 200 mM, it acts as an inhibitory substance, and the synthesis rate of the protein tends to become lower.

The creatine kinase in the reaction solution is a component for continuous synthesis of protein and is added along with creatine phosphate for regeneration of ATP and GTP. The creatine kinase is preferably contained in the reaction solution in a proportion of 1 μg/mL to 1000 μg/mL, more preferably 10 μg/mL to 500 μg/mL, in view of the rate of protein synthesis, When creatine kinase is less than 1 μg/mL, regeneration of sufficient amounts of ATP and GTP becomes difficult. As a result, the rate of protein synthesis tends to become lower, and when creatine kinase exceeds 1000 μg/mL, it acts as an inhibitory substance and the synthesis rate of the protein tends to become lower.

The amino acid component in the reaction solution contains at least 20 kinds of amino acids, i.e., valine, methionine, glutamic acid, alanine, leuicine, phenylalanine, glycine, proline, isoleucine, tryptophan, asparagine, serine, threonine, histidine, aspartic acid, tyrosine, lysine, glutamine, cystine and arginine. The amino acid includes radioisotope-labeled amino acid. Where necessary, modified amino acid may be included. The amino acid component generally contains almost the same amount of various kinds of amino acids.

In the present invention, the above-mentioned amino acid component is preferably contained in the reaction solution in a proportion of 1 μM to 1000 μM, more preferably 10 μM to 200 μM, in view of the rate of protein synthesis. When the amount of the amino acid component is less than 1 μM or more than 1000 μM, the synthesis rate of the protein tends to become lower.

The RNase inhibitor is added to this reaction solution to prevent RNase, which is derived from insect cells present in the extract solution, from undesirably digesting mRNA and tRNA, thereby preventing synthesis of protein, during cell-free protein synthesis of the present invention. It is preferably contained in the reaction solution in a proportion of 0.1 U/μL to 100 U/μL, more preferably 1 U/μL to 10 U/μL. When the amount of the RNase inhibitor is less than 0.1 U/μL, the degradation activity of RNase often cannot be suppressed sufficiently, and when the amount of the RNase inhibitor exceeds 100 U/μL, the protein synthesis reaction tends to be inhibited.

As regards the exogenous mRNA in the reaction solution, so long as the mRNA is not derived from insect cells, the protein (including peptide) that is encoded thereby is not particularly limited, and the mRNA may encode a toxic protein or a glycoprotein. Whether the mRNA contained in the reaction solution is an exogenous mRNA or mRNA derived from an insect cell can be determined by isolating and purifying the mRNA from an extract solution, synthesizing cDNA using a reverse transcriptase, analyzing a base sequence of the obtained cDNA and comparing the base sequences with the base sequences of known exogenous mRNAs.

The exogenous mRNA to be used is not particularly limited as regards the number of bases, and all the exogenous mRNAs need not have the same number of bases as long as they can synthesize the object protein. In addition, as long as the sequences are homologous to the degree that allows synthesis of the object protein, plural bases of each exogenous mRNA may be deleted, substituted, inserted or added.

The exogenous mRNA to be used for the present invention may be a commercially available one or an mRNA obtained by inserting ORF (open reading frame) of the object protein downstream of polyhedron 5′UTR of a commercially available vector, such as pTD1 Vector (manufactured by Shimadzu Corporation), and performing a transcription reaction using the resulting vector. Furthermore, an exogenous mRNA having a cap structure resulting from the addition of methylated ribonucleotide and the like during transcription reaction may be used.

An exogenous mRNA is preferably contained in the reaction solution in a proportion of 5 μg/mL to 2000 μg/mL, more preferably 20 μg/mL to 1000 μg/mL, in view of the protein synthesis rate. When the amount of exogenous mRNA is less than 5 μg/mL or exceeds 2000 μg/mL, the rate of protein synthesis tends to decrease.

The tRNA in the reaction solution contains almost an equal amount each of the tRNAs corresponding to the above-mentioned 20 kinds of amino acids. In the present invention, tRNA is preferably contained in the reaction solution in a proportion of 1 μg/mL to 1000 μg/mL, more preferably 10 μg/mL to 500 μg/mL, in view of the rate of protein synthesis. When the amount of tRNA is less than 1 μg/mL or exceeds 1000 μg/mL, the rate of protein synthesis tends to become lower.

The buffer to be contained in the reaction solution is preferably similar to those used for the aforementioned extract solution of the present invention, and the use of HEPES-KOH (pH 6.5-8.5) is preferable for the same reasons. The buffer is preferably contained in the amount of 5 mM to 200 mM, more preferably 10 mM to 50 mM, from the same perspective as in the aforementioned buffer contained in the extract solution.

The above-mentioned reaction solution preferably contains EGTA. This is because EGTA in extract solution forms a chelate with metal ions therein and inactivates ribonuclease, protease and the like, thereby inhibiting decomposition of the components essential for protein synthesis in the present invention. EGTA is preferably contained in the above-mentioned reaction solution at 0.01 mM to 50 mM, more preferably 0.1 mM to 10 mM, in view of preferable exertion of the above-mentioned decomposition inhibitory ability. When EGTA is contained in less than 0.01 mM, decomposition of essential components cannot be sufficiently suppressed. When it exceeds 50 mM, it tends to inhibit protein synthesis reaction.

In other words, the reaction solution to be used for the cell-free protein synthesis method of the present invention is preferably made to contain the above-mentioned extract solution in a proportion of 30 (v/v) % to 60 (v/v) %, together with 50 mM to 150 mM of potassium salt (e.g., potassium acetate), 0.5 mM to 3 mM of magnesium salt (e.g., magnesium acetate), 0.2 mM to 5 mM of DTT, 0.1 mM to 5 mM of ATP, 0.05 mM to 5 mM of GTP, 10 mM to 100 mM of creatine phosphate, 10 μg/mL to 500 μg/mL of creatine kinase, 10 μM to 200 μM of amino acid component, 1 U/μL to 10 U/μl of RNase inhibitor, 10 μg/mL to 500 μg/mL of tRNA, 20 μg/mL to 1000 μg/mL of exogenous mRNA, 10 mM to 50 mM of buffer (e.g., HEPES-KOH (pH 6.5-8.5)) and 0.3 (v/v) % to 12 (v/v) % of glycerol. In addition, the reaction solution is more preferably made to contain 0.1 mM to 10 mM of EGTA in addition to the above.

The cell-free protein synthesis method of the present invention is performed using the extract solution of the present invention mentioned above in, for example, a conventionally known low temperature incubator. For the reaction, a reaction solution containing the above-mentioned extract solution is generally prepared and used.

The reaction temperature is generally within the range of 10° C. to 40° C., preferably 15° C. to 30° C. When the reaction temperature is lower than 10° C., the protein synthesis rate tends to become lower, and when the reaction temperature exceeds 40° C., the essential components tend to be denatured.

The reaction time is generally 1 to 72 hours, preferably 3 to 24 hours.

The amount of protein synthesized by the cell-free protein synthesis method of the present invention can be measured by activity assay of enzymes, SDS-PAGE, immunoassay and the like.

### EMBODIMENTS

The present invention is explained in detail with reference to embodiments. However, the present invention is not limited to the embodiments.

### Reference Example 1

### Insect Cell Culturing

1.1×10⁸ cells of insect cell Sf21 (manufactured by Invitrogen) were cultured in Sf900 II serum-free media (manufactured by Invitrogen) in a 1-liter Erlenmeyer flask at 27° C., 100 rpm for 64 hours. As a result, the cell count reached 1.6×10⁹ and wet weight 6.4 g.

### Embodiment 1

### Preparation of Tubulin-Free Extract Solution

Insect cells cultured in the afore-described Reference Example 1 were collected and suspended in 8 ml of extraction buffer having the following composition.

### Extraction Buffer Composition

| | | |
|---|---|---|
| 40 | mM | HEPES-KOH (pH 7.9) |
| 100 | mM | Potassium acetate |
| 2 | mM | Magnesium acetate |
| 20% | (v/v) | Glycerol |
| 1 | mM | DTT |
| 2 | mM | EGTA |
| 0.5 | mM | PMSF |

This suspension was rapidly (within 10 seconds) frozen in liquid nitrogen. After freezing sufficiently, the suspension was thawed in an ice-water bath at about 4° C. After completely thawing, the suspension was subjected to centrifugation at 15,000×g, 4° C. for 10 minutes (Himac CR20G manufactured by Hitachi Koki), and supernatant was recovered. The recovered 9 mL of supernatant were added with 190 μL of 100 mM GTP and 90 μL of 2 mM Taxol (registered trademark) (final concentration of 2 mM and 20 μM, respectively) and incubated for 30 minutes at 22° C. This was further subjected to centrifugation at 45000×g at 20° C. for 30 minutes, and the supernatant was recovered.

2.5 mL of the supernatant were applied to PD-10 desalting column (manufactured by GE Healthcare Biosciences) that had been equilibrated with a buffer solution having the following composition for gel filtration.

### Gel Filtration Buffer Solution Composition

| | | |
|---|---|---|
| 40 | mM | HEPES-KOH (pH 7.9) |
| 100 | mM | Potassium acetate |
| 2 | mM | Magnesium acetate |
| 5% | (v/v) | Glycerol |
| 1 | mM | DTT |
| 0.5 | mM | PMSF |

After the application, the supernatant was eluted with a buffer solution for gel filtration (5 mL) and fractions having absorbance at 280 nm of not less than 30 were recovered using a spectrophotometer (Biospec-mini manufactured by Shimadzu Corporation). The recovered fraction was used as the insect cell extract solution.

### Comparison Example 1

### Preparation of Extract Solution Using Conventional Method

First, insect cells that were cultured in afore-described Reference Example 1 were collected and suspended in 8 ml of extraction buffer having the having the following composition.

### Extraction Buffer Composition

| | | |
|---|---|---|
| 40 | mM | HEPES-KOH (pH 7.9) |
| 100 | mM | Potassium acetate |
| 2 | mM | Magnesium acetate |
| 2 | mM | Calcium chloride |
| 20% | (v/v) | Glycerol |
| 1 | mM | DTT |
| 0.5 | mM | PMSF |

This suspension was rapidly (within 10 seconds) frozen in liquid nitrogen. After freezing sufficiently, the suspension was thawed in an ice-water bath at about 4° C. After completely thawing, the suspension was subjected to centrifugation at 15,000×g, 4° C. for 10 minutes (Himac CR20G manufactured by Hitachi Koki), and supernatant was recovered. This was further centrifuged at 45000×g at 4° C. for 30 minutes, and the supernatant was recovered.

2.5 mL of the supernatant were applied to PD-10 desalting column (manufactured by GE Healthcare Biosciences) that had been equilibrated with a buffer solution having the following composition for gel filtration.

### Gel Filtration Buffer Solution Composition

| | | |
|---|---|---|
| 40 | mM | HEPES-KOH (pH 7.9) |
| 100 | mM | Potassium acetate |
| 2 | mM | Magnesium acetate |
| 5% | (v/v) | Glycerol |
| 1 | mM | DTT |
| 0.5 | mM | PMSF |

After the application, the supernatant was eluted with a buffer solution for gel filtration (5 mL) and fractions having absorbance at 280 nm of not less than 30 were recovered using a spectrophotometer (Biospec-mini manufactured by Shimadzu Corporation). The recovered fraction was used as the insect cell extract solution.

### Embodiment 2

### Comparison by Electrophoresis of the Extract Solution of the Present Invention and Conventional Extract Solution

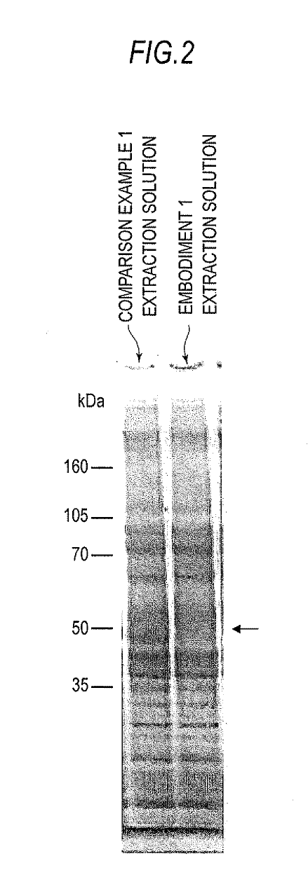

1.25 μL each of the tubulin-free extract solution of the present invention prepared as Embodiment 1 and the conventional extract solution prepared as Comparison Example 1 were separated using 10% SDS-PAGE and stained with CBB. The results are shown in FIG. 2. The thick band (black arrow) detected near 50 kDa is tubulin, and this shows that tubulin is removed in the tubulin-free extract solution.

### Reference Example 2

mRNA Preparation

mRNA that codes for β-galactosidase was prepared in the following manner. This was done so that the protein synthesis ability of the tubulin-free extract solution of the present invention can be evaluated using protein that can be purified without any problems when synthesized using an extract solution that was prepared using a conventional method.

Using control DNA (linear DNA comprising expression vector pTD1 with β-galactosidase gene incorporated therein) included in transdirect insect cells (manufactured by Shimadzu Corporation) and an mRNA synthesis kit, T7 RiboMAX™ Express Large Scale RNA Production System (manufactured by Promega), mRNA was synthesized following the product protocol. The obtained reaction solution following the completion of the synthesis was applied to Nick column (manufactured by GE Healthcare Biosciences) and eluted with 400 μL of sterilized water. The eluted fraction was recovered, potassium acetate was added to achieve a final concentration of 0.3 M, and ethanol precipitation was conducted. For quantification of the synthesized mRNA, absorbance at 260 nm was measured. As a result, about 450 μg of mRNA was synthesized by a 100 μL scale reaction.

### Embodiment 3

### Cell-Free Synthesis of β-Galactosidase Using Tubulin-Free Extract Solution

Using the tubulin-free extract solution of the present invention of Embodiment 1 and mRNA of Reference Example 2, reaction solution having the following composition was prepared, and protein synthesis was performed using a cell-free system.

### Reaction Solution Composition

| | | |
|---|---|---|
| 50 | (v/v) % | Tubulin-free extract solution of Embodiment 1 |
| 40 | mM | HEPES-KOH (pH7.9) |
| 100 | mM | Potassium acetate |
| 1.5 | mM | Magnesium acetate |
| 2 | mM | DTT |
| 0.25 | mM | ATP (manufactured by Sigma) |
| 0.1 | mM | GTP (manufactured by Sigma) |
| 20 | mM | Creatine phosphate |
| 200 | μg/mL | Creatine kinase |
| 80 | μM | Amino acids (20 types) (manufactured by Sigma) |
| 0.1 | mM | EGTA |
| 200 | μg/mL | tRNA (Roche Diagnostics) |
| 2.5 | (v/v) % | Glycerol (insect cell extract solution-derived) |
| 0.25 | mM | PMSF (insect cell extract solution-derived) |
| 320 | μg/mL | mRNA(codes for β-galactosidase gene) |

A low-temperature aluminum block incubator, the MG-1000, was used as the reaction device. The reaction was performed using a reaction solution quantity of 25 μL, reaction temperature of 25° C. and reaction time of 5 hours. The activity of the synthesized β-galactosidase was quantified using a β-galactosidase assay kit (manufactured by Promega). β-galactosidase activity was measured by preparing a calibration curve using a spectrophotometer (Biospec-mini manufactured by Shimadzu Corporation) following its instruction manual.

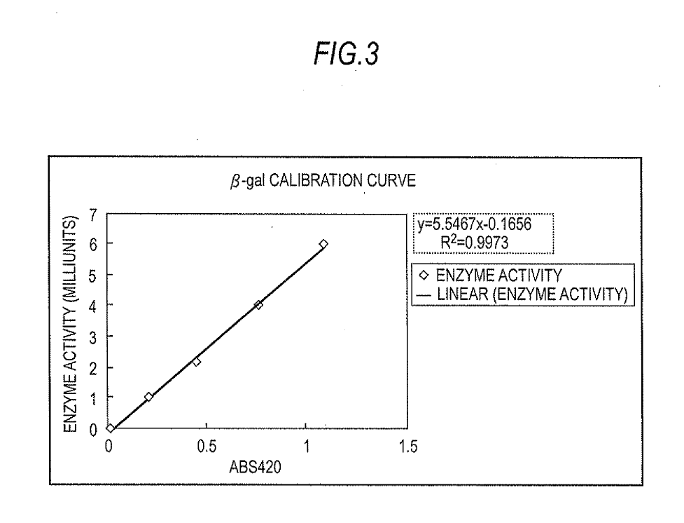

FIG. 3 shows the prepared calibration curve. Because ABS₄₂₀ of a 10-fold diluted sample was 0.840, the enzyme activity in light of the calibration curve was calculated to be 44.9 UI mL.

### Comparison Example 2

### Cell-Free Synthesis of β-Galactosidase Using Conventional Extract Solution

Using the extract solution of Comparison Example 1 prepared using the conventional method and mRNA of Reference Example 2, β-galactosidase was synthesized and its activity calculated. (This meant that the procedure was the same as in Embodiment 3 except for the use of the extract solution of Comparison Example 1 prepared using the conventional method instead of the tubulin-free extract solution of Embodiment 1 prepared using the method of the present invention.)

Because ABS₄₂₀ of the 10-fold diluted sample was 0.818, the enzyme activity was calculated to be 43.7 U/mL.

This showed that the tubulin-free extract solution had a protein synthesis ability that was substantially comparable to that of a conventional extract solution.

### Reference Example 3

### Expression Vector Construction

To evaluate the effects of tubulin removal using a protein whose purification is difficult when synthesized using an extract solution that is prepared by the conventional method, an expression vector that coded for the OCT4 gene was prepared using DNA fragments having the following sequences.

Sequence No. 1:
G8-FLAG-F
GGGAATTCGGTACCGGATCCGGTGGAGGTGGAGGTGGAGGTGGAGACT
ACAAGGATGACGATGACAAGTAATCTAGAGC
Sequence No. 2:
G8-FLAG-R
GCTCTAGATTACTTGTCATCGTCATCCTTGTAGTCTCCACCTCCACCT
CCACCTCCACCGGATCCGGTACCGAATTCCC
Sequence No. 3:
T7 promoter
GCAGATTGTACTGAGAGTG
Sequence No. 4:
OCT-Fw
ATGGCGGGACACCTGG
Sequence No. 5:
OCT-Rv
GGGAATTCGTTTGAATGCATGGGAGAGC

To introduce a purification FLAG tag in expression vector pTD1 that is included in the transdirect insect cells (manufactured by Shimadzu Corporation), a DNA fragment (G8-FLAG-F) having the base sequence described in sequence No. 1 and DNA fragment (G8-FLAG-R) having the base sequence described in sequence No. 2 were mixed, annealed and then digested using EcoRI and XbaI. Separately from this digestion, pTD1 was digested with EcoRI and XbaI. Next, using Ligation-Convenience Kit (manufactured by Nippon Gene), these DNA fragments were ligated. Following the ligation, E. coli, DH5α (manufactured by Nippon Gene), was transformed. Plasmid DNA was prepared from the transformed E. coli by alkali-SDS methods and was subjected to a sequencing reaction (96° C., 10 seconds, 50° C., 5 seconds, 60° C., 4 minutes, 30 cycles) using primer (T7 promoter) having a base sequence described in in sequence No. 3 and Big Dye Terminator Cycle Sequencing FS (Applied Biosystems). This reaction solution was applied to ABI PRISM 310 Genetic Analyzer (manufactured by Applied Biosystems), and the base sequence was analyzed.

A plasmid having a spacer sequence comprising 8 glycines and the FLAG tag sequence inserted downstream of the multiple cloning site of the pTD1 vector was named as pTD1-FLAG.

Using pF1KB7614 (manufactured by Promega) as a template, primer OCT-Fw having the base sequence described in sequence No. 4 and primer OCT-Rv having the base sequence described in sequence No. 5 and KOD plus (manufactured by Toyobo), 30 cycles of PCR were performed as follows: 97° C.—15 seconds, 50° C.—30 seconds, and 68° C.—60 seconds. DNA fragment was purified by ethanol precipitation and then digested with EcoRI. Separately from this digestion, the afore-mentioned pTD1-FLAG was digested by EcoRV and EcoRI. Next, using Ligation-Convenience Kit (manufactured by Nippon Gene), these DNA fragments were ligated. Following the ligation, E. coli, DH5α, (manufactured by Nippon Gene), was transformed. Plasmid DNA was prepared from the transformed E. coli by alkali-SDS methods, and was subjected to a sequencing reaction (96° C., 10 seconds, 50° C., 5 seconds, 60° C., 4 minutes, 30 cycles) using primer (T7 promoter) having a base sequence described in in sequence No. 3 and Big Dye Terminator Cycle Sequencing FS (manufactured by Applied Biosystems). This reaction solution was applied to ABI PRISM 310 Genetic Analyzer (manufactured by Applied Biosystems), and the base sequence was analyzed.

The expression vector that codes for the OCT4 gene at multiple coning sites of pTD1-FLAG was named as pTD1-FLAG-OCT4.

### Reference Example 4

mRNA Preparation

Using pTD1-FLAG-OCT4 of Reference Example 3 and an mRNA synthesis kit, T7 RiboMAX™ Express Large Scale RNA Production System (manufactured by Promega), mRNA was synthesized following the product protocol. The obtained reaction solution following the completion of the synthesis was applied to Nick column (manufactured by GE Healthcare Biosciences) and eluted with 400 μL of sterilized water. The eluted fraction was recovered, potassium acetate was added to achieve a final concentration of 0.3 M, and ethanol precipitation was conducted. For quantification of the synthesized mRNA, absorbance at 260 nm was measured. As a result, about 604 μg of mRNA was synthesized by a 100 μL scale reaction.

### Embodiment 4

### Cell-Free Synthesis of OCT4 Using Tubulin-Free Extract Solution and Affinity Purification

Using the tubulin-free extract solution of Embodiment 1 and the mRNA of Reference Example 4, reaction solution with the following composition was prepared, and protein synthesis was performed using a cell-free system.

### Reaction Solution Composition

| | | |
|---|---|---|
| 50 | (v/v) % | Tubulin-free extract solution obtained in Embodiment 1 |
| 40 | mM | HEPES-KOH (pH7.9) |
| 100 | mM | Potassium acetate |
| 1.5 | mM | Magnesium acetate |
| 2 | mM | DTT |
| 0.25 | mM | ATP (manufactured by Sigma) |
| 0.1 | mM | GTP (manufactured by Sigma) |
| 20 | mM | Creatine phosphate |
| 200 | μg/mL | Creatine kinase |
| 80 | μM | Amino acids (20 types) (manufactured by Sigma) |
| 0.1 | mM | EGTA |
| 200 | μg/mL | tRNA (Roche Diagnostics) |
| 2.5 | (v/v) % | Glycerol (insect cell extract solution-derived) |
| 0.25 | mM | PMSF (insect cell extract solution-derived) |
| 320 | μg/mL | mRNA (codes for OCT4 gene) |

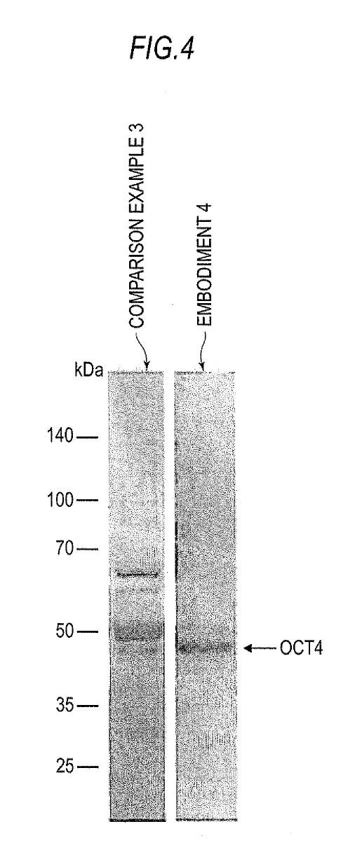

A low-temperature aluminum block incubator, the MG-1000, was used as the reaction device. The reaction was performed using a reaction solution quantity of 1000 μL, reaction temperature of 25° C. and reaction time of 5 hours. After the completion of the reaction, the reaction solution was subjected to centrifugation of 15000×g, 25° C., 15 minutes, and its supernatant was desalted by applying to PD-10 (manufactured by GE Healthcare Biosciences) that had been equilibrated with 50 mM Tris-HCl, pH8.0 and 150 mM NaCl (Buffer A). The eluted solution was applied to an open column (0.5 mL) of Anti-FLAG^{□} M2 Agarose from mouse (manufactured by SIGMA) that had been equilibrated with Buffer A. The column was washed with 1.0 mL of Buffer A. After repeating this washing for a total of 5 times, Buffer A containing 100 μg/mL of FLAG Peptide (manufactured by SIGMA) was added and allowed to elute. The eluted solution was concentrated to 50 μL by spin-type ultrafiltration. After separating 5 μL of the eluted solution using 10% SDS-PAGE, staining was done using CBB. FIG. 4 shows the results. What was detected was substantially a single band.

### Comparison Example 3

### Cell-Free Synthesis of OCT4 Using Conventional Extract Solution and Affinity Purification

Using the extract solution of Comparison Example 1 prepared using the conventional method and mRNA of Reference Example 4, OCT4 was synthesized in the same way as in Embodiment 4. (This meant that the procedure was the same as in Embodiment 4 except for the use of the extract solution prepared using the conventional method of Comparison Example 1 instead of the tubulin-free extract solution of Embodiment 1 prepared using the method of the present invention.) Affinity purification was then performed.

Just as with Embodiment 4, after separating using 10% SDS-PAGE, staining was done using CBB. FIG. 4 shows the results. Even though OCT4 was detected, tubulin was detected as the main band, and a plurality of bands other than tubulin was also detected.

A comparison of afore-described Embodiment 4 and Comparison Example 3 showed that, with the tubulin-free extract solution of the present invention, purification of a high purity is possible even when a conventional extract solution is used so that the protein may have a lower purification purity due to the mixed presence of tubulin.

### Sequence Table Free Text

Sequence Nos. 1 to 5 show synthesized oligonucleotides.

What is claimed is:

1. A method for preparing cell extract solution for cell-free protein synthesis comprising:
obtaining extraction treated material of cultured cells by subjecting cultured cells to an extraction treatment using an extraction buffer;
obtaining a reaction mixture by polymerizing tubulin derived from said cultured cells included in said extraction treated material by subjecting said extraction treated material to a tubulin polymerization reaction; and
obtaining cell extract solution for cell-free protein synthesis by subjecting said reaction mixture to removal of polymerized tubulin and buffer exchange.

2. The method according to claim 1 wherein:
in the afore-described step for obtaining extraction treated material of cultured cells, after said extraction treatment, extraction mixture that is obtained by said extraction treatment is subjected to centrifugation under the conditions of 10,000×g to 50,000×g for 1 to 60 minutes; and
a supernatant that is obtained by said centrifugation is obtained as said extraction treated material of cultured cells.

3. The method according to claim 1 wherein said extraction treatment is performed by rapidly freezing said cultured cells that are suspended in said extraction buffer and thawing said frozen cultured cells.

4. The method according to claim 1 wherein said tubulin polymerization reaction is performed using taxane compounds and/or epothilone compounds.

5. The method according to claim 1 wherein said cultured cells are cultured insect cells.

6. The method according to claim 5 wherein said cultured insect cells are Trichoplusia ni ovum-derived cultured cells and/or Spodoptera frugiperda ovary cell-derived cultured cells.

7. A reagent kit for cell-free protein synthesis that includes cell extract solution for cell-free protein synthesis wherein the cell extract solution have been prepared using a method described in claim 1.

8. A method for cell-free protein synthesis that uses cell extract solution for cell-free protein synthesis wherein the cell extract solution have been prepared using a method described in claim 1.
